(19) United States
(12) Patent Application Publication
(10) Pub. No.: US 2016/0296637 A1
(43) Pub. Date: Oct. 13, 2016

(54) Method for improving survival after radiation exposure using mesenchymal stem cells as vehicles for extracellular superoxide dismutase delivery

(71) Applicants: Weiwen Deng, Cupertino, CA (US); Aly S Abdel-Mageed, Ada, MI (US); Anthony J. Senagore, Cleveland, OH (US); Rick V Hay, Ada, MI (US)

(72) Inventors: Weiwen Deng, Cupertino, CA (US); Aly S Abdel-Mageed, Ada, MI (US); Anthony J. Senagore, Cleveland, OH (US); Rick V Hay, Ada, MI (US)

(21) Appl. No.: 14/684,370

(22) Filed: Apr. 11, 2015

(51) Int. Cl. A61K 48/00; C12N 15/86

(52) U.S. Cl. CPC A61K 48/0008; C12N 15/86; A61K 48/0075; A61K 48/005; C12N 2710/10042

(57) ABSTRACT

A method for improving survival after radiation exposure using mesenchymal stem cells as vehicles to deliver extracellular superoxide dismutase is provided. The invention encompasses a method to improve survival after radiation exposure that harnesses the power of migration of mesenchymal stem cells to radiation injured tissues and adenovirus-mediated extracellular superoxide dismutase gene therapy for oxidative stress caused by radiation exposure. The invention relates to methods for producing mesenchymal stem cells secreting biologically active extracellular superoxide dismutase. Methods for treating a human patient after radiation exposure by intravenous administration of mesenchymal stem cells secreting biologically active extracellular superoxide dismutase are also disclosed.

### CROSS-REFERENCE TO RELATED APPLICATIONS

U.S. Provisional Application No. 61/981,207 was filed on Apr. 18, 2014. This application is a divisional of U.S. Ser. No. 2011/0225661, filed Sep. 15, 2011, now abandoned, which claims priority under 35 U.S.C. sctn. 119(e) to U.S. Provisional application Ser. No. 12/677,001, filed Jun. 26, 2009, both of which are incorporated herein by reference in their entirety.

### BACKGROUND OF THE INVENTION

Formation of superoxide anion (O₂⁻) after ionizing radiation is a major determinant of the lethality of whole-body radiation exposure (Greenberger and Epperly, in: Columbus F, editor. Progress in Gene Therapy. New York, N.Y.: Nova Science Publications; 2005. p. 110-118. Fliedner et al., Curr Opin Hematol 2006; 13:436-444; Greenberger, Pharmacogenomics 2006; 7:1141-1145). Extracellular superoxide dismutase (ECSOD) is a potent antioxidant enzyme catalyzing the dismutation of O₂⁻ and ECSOD has already been used in gene therapy of diseases involving oxidative stress (Bivalacqua et al., J Sex Med 2005; 2:187-197). Therefore, the enhancement of ECSOD is a promising approach for improving survival after radiation exposure. An attractive method of increasing ECSOD delivery is the use of gene transfer technology to locally increase ECSOD gene expression. However, in vivo administration of non-viral vectors or viral vectors usually leads to low levels of gene transfer, and as the transgene is randomly expressed in almost all cell types, adverse effects can occur (Deng et al., Am J Physiol Cell Physiol. 2003; 285:C1322-9).

Until the present invention, a need has existed to develop an improved ECSOD gene therapy method to improve survival after radiation exposure. The present invention fulfills this need.

### BRIEF SUMMARY OF THE INVENTION

The present invention encompasses a method of improving survival after radiation exposure in human. The method comprises bone marrow mesenchymal stem cell-based extracellular superoxide dismutase gene delivery system. In a preferred aspect, the system is composed of human mesenchymal stem cell (MSC) and human extracellular superoxide dismutase (ECSOD).

In another aspect, the method comprises producing mesenchymal stem cell that secretes biologically active ECSOD enzyme (ECSOD-MSC) for improving survival after radiation exposure in human. In a preferred aspect, the method comprises adenoviral-mediated ECSOD gene transfer into MSC. The adenoviral-transduced mesenchymal stem cell produced by the method secretes biologically active ECSOD enzyme. The method is composed of human mesenchymal stem cell (MSC) and human extracellular superoxide dismutase (ECSOD).

The invention also includes a method of intravenous administration of mesenchymal stem cell that secretes biologically active ECSOD enzyme (ECSOD-MSC) for improving survival after radiation exposure in human. In a preferred aspect, the method comprises treating a human patient after radiation exposure with intravenous administration of ECSOD-MSCs, wherein ECSOD-MSCs migrate to radiation-injured tissues such as bone marrow and gastrointestinal tract after intravenous administration, locally secrete biologically active ECSOD enzyme, and the secreted ECSOD enzyme scavenges superoxide anion (O₂⁻) in radiation-injured tissues, thereby improving survival of human patients with radiation exposure. In the method, mesenchymal stem cell (MSC) functions as a vehicle to deliver ECSOD enzyme to radiation-injured tissues. The method is composed of human mesenchymal stem cell (MSC) and human extracellular superoxide dismutase (ECSOD). In a preferred aspect, the radiation exposure is γ radiation from nuclear war, nuclear accident, nuclear terrorism, radiation diagnosis, radiation therapy, radiological emergencies, and space travel.

### BRIEF DESCRIPTION OF THE DRAWINGS

The foregoing summary and the following detailed description of the invention will be better understood when read in conjunction with the appended drawings. It should be understood that the invention is not limited to the precise arrangements and instrumentalities shown in the drawings.

FIG. 1, comprising FIGS. 1A-1D, demonstrates that intravenous administration of mouse MSCs (mMSCs) that secretes biologically active ECSOD enzyme (ECSOD-mMSCs) improves survival of irradiated mice. FIG. 1A depicts phenotype of mMSCs by flow cytometry. FIG. 1B shows secretion of biologically active ECSOD by Ad5CMVECSOD-transduced mMSCs (ECSOD-mMSCs). FIG. 1C shows expression of nuclear-targeted β-galactosidase by Ad5CMVntlacZ-transduced mMSCs (ntlacZ-mMSCs). FIG. 1D shows that intravenous administration of ECSOD-mMSCs improves survival of irradiated mice.

FIG. 2 shows the effect of ¹³⁷Cs γ radiation on cell counts of peripheral blood in mice. Five-week-old female BALB/c mice were given 9 Gy total body γ irradiation from a ¹³⁷Cs source. Seven days later, the mice were sacrificed. Peripheral blood was analyzed for complete blood count (CBC) using the fully automated instrument VetScan HM2 Hematology System (Abaxis Inc., Union city, CA). The counts of white blood cell (WBC), red blood cell (RBC), and platelet in whole blood were then compared with those of unirradiated mice. Data were expressed as mean±SEM (n=4) and statistically analyzed using t test.

FIG. 3 shows the effect of intravenous administration of ECSOD-mMSCs on mouse body weight loss after radiation exposure. Five-week old, female BALB/c mice were given 9 Gy total body γ irradiation from a ¹³⁷Cs source. After 24 hours, the animals were given a tail vein injection of 0.5×10⁶ ECSOD-mMSCs. Mouse body weight was then monitored daily for 35 days. Each value represented mean±SEM. Five-week old, female BALB/c healthy unirradiated mice were used as control.

FIG. 4, comprising FIGS. 4A-4E, demonstrates that intravenous administration of ECSOD-mMSCs extends lifespan, retards cataract formation, and prevents carcinogenesis in irradiated mice. FIG. 4A shows secretion of biologically active ECSOD by ECSOD-mMSCs at various time intervals after adenoviral transduction. FIG. 4B shows expression of nuclear-targeted β-galactosidase by ntlacZ-mMSCs at various time intervals after transduction. FIG. 4C is a Kaplan-Meier survival curve showing that intravenous administration of ECSOD-mMSCs extends lifespan of irradiated mice. P=0.003 by logrank test. FIG. 4D shows that intravenous administration of ECSOD-mMSCs retards cataract formation of irradiated mice. Picture was taken 172 days after irradiation. FIG. 4E shows no evidence of carcinogenesis in irradiated mice after intravenous administration of ECSOD-mMSCs. Picture was taken 449 days after irradiation.

FIG. 5 demonstrates no new bone formation in irradiated mice treated with ECSOD-mMSCs by SPET CT.

FIG. 6, comprising FIGS. 6A-6F, demonstrates that human MSCs (hMSCs) can be genetically modified with adenoviral vector to secrete high levels of biologically active ECSOD. FIG. 6A shows colony-forming unit (CFU) of hMSCs in 14-day culture of 1×10⁵ human bone marrow mononuclear cells (BM-MNCs). The 14-day culture of 1×10⁵ human peripheral blood mononuclear cells (PB-MNCs) was used as a negative control. FIG. 6B shows quantification of CFU of hMSCs in 14-day culture of 1×10⁵ human BM-MNCs. FIG. 6C depicts phenotype of hMSCs by flow cytometry. FIG. 6D is a spectral karyotyping (SKY) cytogenetic analysis showing hMSCs with a normal diploid pattern of male human origin (46, XY). FIG. 6E shows dose-dependent expression of nuclear-targeted β-galactosidase by Ad5CMVntlacZ-transduced hMSCs (ntlacZ-hMSCs). FIG. 6F shows dose-dependent, high-level secretion of biologically active ECSOD by Ad5CMVECSOD-transduced hMSCs (ECSOD-hMSCs).

FIG. 7 shows the grand strategy of our novel method for improving survival after radiation exposure using mesenchymal stem cells as vehicles for extracellular superoxide dismutase delivery

### DETAILED DESCRIPTION OF THE INVENTION

The invention herein described demonstrates another way to treat a human patient for improving survival after radiation exposure. The method involves intravenous administration of human mesenchymal stem cells (MSCs) that secrete biologically active extracellular dismutase (ECSOD) enzyme (ECSOD-hMSCs) to a human patient with radiation exposure, wherein ECSOD-hMSCs migrate to radiation-injured tissues such as bone marrow and gastrointestinal tract after intravenous administration, locally secrete biologically active ECSOD enzyme, and the secreted ECSOD enzyme scavenges superoxide anion (O₂⁻) in radiation-injured tissues, thereby improving survival of human patients with radiation exposure.

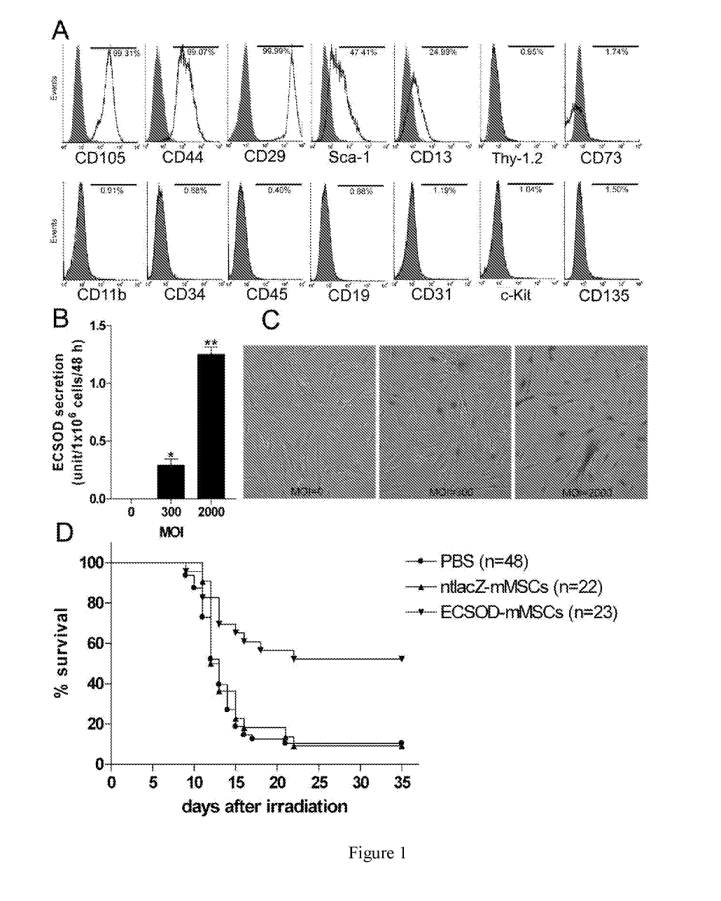

The invention comprises and utilizes the discovery that intravenous administration of mouse MSCs that secrete biologically active ECSOD enzyme (ECSOD-mMSCs) improved survival of irradiated mice from 10% to 52% (Abdel-Mageed et al., Blood 113: 1201-1203). See FIG. 1D.

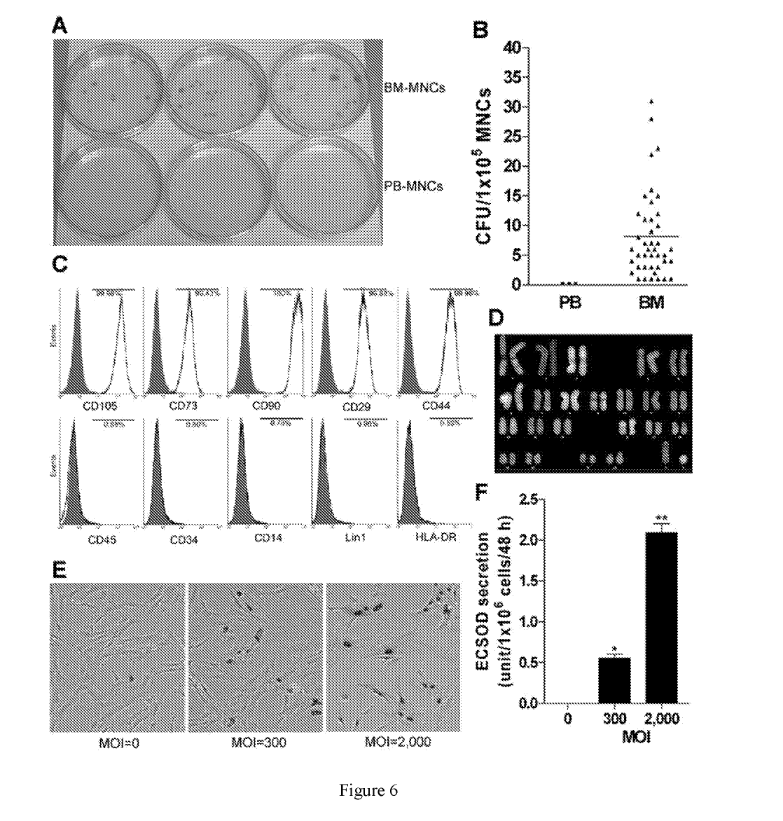

The invention includes a method of improving survival after radiation exposure. Embodiments of the method of the invention are described in the Examples section herein. Generally, bone marrow cells are isolated from a human donor or the patient him/herself, human mesenchymal stem cells (hMSCs) are obtained therefrom, and the hMSCs are subsequently expanded in vitro on flasks or dishes using standard cell culture techniques, e.g., as described in the materials and methods section of the Examples. See FIGS. 6A-D.

To produce hMSCs that secrete biologically active ECSOD enzyme (ECSOD-hMSCs) by adenoviral transduction, hMSCs are transduced with an effective amount of adenovirus containing ECSOD for a period of time. The amount of the adenovirus and the length of time may vary according to the precise method being contemplated and should not be construed as limiting the invention in any way. After adenoviral transduction, the cells are further cultured for a period of time and the culture supernatant is prepared for superoxide dismutase (SOD) enzyme activity assay to determine secretion of ECSOD by the cells. High level secretion of biologically active ECSOD enzyme is evident within about 2 days. See FIG. 6F.

The invention further includes a method of treating a human patient after radiation exposure to improve survival by intravenous administering ECSOD-hMSCs of the invention to the circulation system of the patient. hMSCs from a human donor or the patient him/herself are transduced with an effective amount of adenovirus containing ECSOD for a period of time to produce ECSOD-hMSCs. After adenoviral transduction, an effective amount of ECSOD-hMSCs are intravenously administered to a human patient at a period of time after radiation exposure. The amount of ECSOD-hMSCs and the length of time after radiation exposure may vary according to the precise method being contemplated and should not be construed as limiting the invention in any way. The time of ECSOD-hMSCs treatment, e.g. once, twice, three times, may vary according to the precise method being contemplated and should not be construed as limiting the invention in any way.

### DEFINITIONS

As used herein, radiation exposure is meant to refer to exposure to γ radiation from nuclear war, nuclear accident, nuclear terrorism, radiation diagnosis, radiation therapy, radiological emergencies, and space travel.

As used herein, mesenchymal stem cell (MSC) and mesenchymal stem cells (MSCs) are used interchangeably and are meant to refer to the small fraction of cells in bone marrow which can serve as precursors of osteocytes, chondrocytes, and adipocytes and which are isolated from bone marrow by their ability to adhere to plastic dishes. Mesenchymal stem cells may be derived from any animal. In some embodiments, mesenchymal stem cells are derived from primates, preferably humans.

The term “mesenchymal stem cells as vehicles” as used herein should be construed to mean the power of mesenchymal stem cells to migrate to radiation injured tissues after intravenous administration is harnessed for adult stem cell-based gene therapy after radiation exposure.

### Examples

The invention is further described in detail by reference to the following experimental examples. These examples are provided for purposes of illustration only. They are not intended to be limiting unless otherwise specified. Thus the invention should not be construed as being limited to the following examples. The invention should be construed to encompass any and all variations, which become evident as a result of the teaching provided herein.

The materials and methods are now discussed.

### Adenoviral Vectors

Two replication-deficient recombinant adenoviruses were used: (1) Ad5CMVECSOD carries human ECSOD gene under the control of cytomegalovirus (CMV) promoter; (2) Ad5CMVntlacZ carries nuclear-targeted β-galactosidase gene ntlacZ under the control of CMV promoter. Both adenoviral vectors were purchased from University of Iowa Gene Transfer Vector Core (Iowa City, Iowa) (Chu et al; Circ Res. 2003; 92:461-468).

Isolation and Ex Vivo Expansion of Mouse MSCs (mMSCs)

mMSCs were isolated by their adherence to tissue-culture plastic as previously described (Deng et al., Am J Physiol Cell Physiol. 2003; 285: C1322-9; Sun et al., Stem Cells. 2003; 21:527-535; Peister et al., Blood. 2004; 103:1662-1668; Abdel-Mageed et al., Blood. 2009; 113: 1201-1203). Briefly, 6-week-old female BALB/c mice (The Jackson Laboratory, Bar Harbor, Me.) were euthanized with CO₂ and femurs and tibias were removed. Both ends of the bones were cut and bone marrow was flushed out using a 18-gauge needle and culture medium for mMSCs (MEM-α containing 20% fetal bovine serum, 100 u/ml penicillin, 100 μg/ml streptomycin, 250 ng/ml amphotericin B, and 2 mM L-glutamine). The bone marrow cells were filtered through a cell strainer with 70-μm nylon mesh, and the cells from one mouse were plated in a T75 flask. The cells were incubated at 37° C. with 5% humidified CO₂, and mMSCs were isolated by their adherence to tissue culture plastic. Fresh culture medium was added and replaced every 2-3 days. The adherent mMSCs were grown to 90% confluency, harvested with 0.25% trypsin and 1 mM EDTA for 2 minutes at 37° C., and diluted 1:3 for ex vivo expansion.

Isolation and Ex Vivo Expansion of Human Mesenchymal Stem Cells (hMSCs)

hMSCs were isolated by their adherence to tissue culture plastic as previously described (Prockop, Science. 1997; 276:71-74; Pittenger et al., Science. 1999; 284:143-147; Deng et al., Biochem Biophys Res Commun. 2001; 282:148-152). Briefly, 5 ml bone marrow aspirate from healthy donors was diluted 1:1 with Hanks' balanced salt solution (HBSS, GIBCO Invitrogen Corp.) and layered over 7.5 ml Fico/Lite-LymphoH (Atlanta Biologicals). After centrifugation at 400 g for 30 min, the mononuclear cell layer was recovered from the gradient interface and washed twice with HBSS. The cells were suspended in 10 ml complete culture medium for hMSCs (MEM-α containing 20% fetal bovine serum, 100 u/ml penicillin, 100 μg/ml streptomycin, and 2 mM L-glutamine). All of the cells were then plated in one T75 flask (Falcon) and incubated at 37° C. with 5% humidified CO₂. Fresh culture medium was added and replaced every 2-3 days to remove non-adherent cells. The adherent hMSCs were grown to 90% confluency over about 14 days. The cells were harvested with 0.25% trypsin/1 mM EDTA (Atlanta Biologicals) for 2 min at 37° C., diluted 1:3, re-plated in T75 flasks (Falcon), and again grown to 90% confluency. hMSCs were then harvested with 0.25% trypsin/1 mM EDTA (Atlanta Biologicals) and diluted 1:3 per passage for further ex vivo expansion.

Phenotype Analysis of mMSCs

Flow cytometric analysis of phenotype of mMSCs was conducted as previously described (Sun et al, Stem Cells 2003; 21:527-535. Peister et al, Blood 2004; 103:1662-1668; Abdel-Mageed et al., Blood. 2009; 113:1201-1203) on ex vivo expanded mMSCs to determine the expression of CD11b, CD13, CD19, CD29, CD31, CD34, CD44, CD45, CD73, CD90 (Thy-1.2), CD105, CD117 (c-Kit), CD135, and Sca-1. Histograms show the relative intensity of mMSCs for various cell-surface antigens. Numbers indicate the percentage of cells in the population whose staining intensity with the specific antibody (white) was greater than that with the respective isotype control (gray).

Phenotype Analysis of hMSCs

Flow cytometric analysis of phenotype of hMSCs was conducted as previously described (Prockop, Science. 1997; 276:71-74. Pittenger et al., Science. 1999; 284:143-147; Dominici et al., Cytotherapy. 2006; 8:315-317) on ex vivo expanded hMSCs to determine the expression of CD14, CD29, CD34, CD44, CD45, CD73, CD90, CD105, Lineage cocktail 1 (Lin1, i.e. CD3, CD14, CD16, CD19, CD20, and CD56), and HLA-DR. Histograms show the relative intensity of hMSCs for various cell-surface antigens. Numbers indicate the percentage of cells in the population whose staining intensity with the specific antibody (white) was greater than that with the respective isotype control (gray).

### Colony-Forming Unit (CFU) Assay

Analysis of CFU of hMSCs in bone marrow mononuclear cells (BM-MNCs) or peripheral blood mononuclear cells (PB-MNCs) was conducted as previously described (Pittenger et al., Science. 1999; 284:143-147). Briefly, 5 ml bone marrow aspirate or peripheral blood from healthy donors was diluted 1:1 with Hanks' balanced salt solution (HBSS, GIBCO Invitrogen Corp.) and layered over 7.5 ml Fico/Lite-LymphoH (Atlanta Biologicals). After centrifugation at 400 g for 30 min, the mononuclear cell layer was recovered from the gradient interface and washed twice with HBSS. The cells were suspended in 10 ml complete culture medium for hMSCs [MEM-α (Atlanta Biologicals); 20% fetal bovine serum (FBS, GIBCO Invitrogen Corp.); Penicillin-Streptomycin (100 units/ml penicillin and 100 μg/ml streptomycin, Atlanta Biologicals); and 2 mM L-glutamine (GIBCO Invitrogen Corp.)]. The cells were then counted with a hematocytometer (Fischer Scientific) and cell viability was determined by Trypan blue exclusion method. To conduct CFU assay, 1×10⁵ BM-MNCs or PB-MNCs in 10 ml culture medium were plated in a 10-cm culture dish (Falcon) and cultured for 14 days with no culture medium change. The culture dish was washed briefly with PBS and stained with 3 ml 3% crystal violet in methanol (Sigma) at room temperature for 15 min. The dish was then washed with dH₂O and all the colonies of hMSCs were counted. Triplicate dishes were used and the average number of cell colony per dish per 1×10⁵ MNCs was counted as CFU value.

Cytogenetic Analysis of hMSCs

Spectral karyotyping (SKY) analysis of cytogenetics of hMSCs was conducted as previously described (Peterson et al., Methods Mol Biol. 2008; 438:197-204) to determine whether cytogenetic abnormalities were acquired after ex vivo expansion of hMSCs. Briefly, hMSCs were arrested in metaphase after the addition of 75 ng/ml colcemid (Sigma) for 1.5 hours. The cells were harvested according to standard cytogenetic procedures involving hypotonic treatment in 75 mM potassium chloride and fixation in 3:1 methanol/acetic acid (Sigma). Metaphase slides were prepared over a humidified waterbath for proper chromosome spreading. Six microliters of denatured SkyPaint probe [Applied Spectral Imaging (ASI), Migdal Ha'Emek, Israel] was added to each denatured metaphase slide, which then was covered by a glass coverslip and incubated overnight in a 37° C. humidified chamber. Slide pretreatment and posthybridization washes were performed according to the standard supplied protocol (ASI). Image acquisition was performed with a COOL-1300 SpectraCube camera (ASI) mounted on an Olympus BX51 microscope with a SKY optical filter (ASI). Twenty metaphases were analyzed using the SKYView EXPO v2.1.1 software (ASI).

### Adenoviral Transduction of MSCs to Secrete Biologically Active ECSOD

MSCs were transduced with Ad5CMVECSOD as previously described (Deng et al., Stem Cells. 2004; 22:1279-1291; Baber et al., Am J Physiol Heart Circ Physiol. 2007; 292: H1120-1128; Abdel-Mageed et al., Blood. 2009; 113:1201-1203). Briefly, mMSCs or hMSCs were plated at a density of 1×10⁴ cells/cm² in 6-well plates or T75 flasks (Falcon) and incubated overnight. The cells were counted and then exposed to fresh culture medium containing Ad5CMVECSOD at 0, 300, or 2,000 multiplicities of infection (MOI, defined as plaque-forming unit/cell) for 48 hours. Virus-containing culture medium was discarded, cells were washed 3 times with phosphate-buffered saline (PBS), and fresh culture medium was added. Cells were then counted, cultured for 48 hours, and culture supernatant was collected. The culture supernatant was then assayed for the secretion of biologically active ECSOD by Ad5CMVECSOD-transduced mMSCs or hMSCs (ECSOD-mMSCs or ECSOD-hMSCs) using a superoxide dismutase (SOD) activity assay kit (Cayman Chemical Company, Ann Arbor, Mich.). Ad5CMVntlacZ-transduced mMSCs or hMSCs (ntlacZ-mMSCs or ntlacZ-hMSCs) and wild type mMSCs or hMSCs were used as controls. Data were expressed as mean±SEM (n=3) and analyzed statistically using a one-way analysis of variance (ANOVA) followed by post-hoc analysis with Tukey test. *P<0.001 versus MOI 0; **P<0.001 versus MOI 0 or 300.

### Adenoviral Transduction of MSCs to Express β-Galactosidase

MSCs were transduced with Ad5CMVntlacZ as previously described (Deng et al., Am J Physiol Cell Physiol. 2003; 285: C1322-1329; Deng et al., Stem Cells. 2004; 22:1279-1291; Deng et al., Life Sci 2006; 78:1830-1838; Baber et al., Am J Physiol Heart Circ Physiol. 2007; 292: H1120-1128; Bivalacqua et al., Am J Physiol Heart Circ Physiol. 2007; 292:H1278-1290; Abdel-Mageed et al., Blood. 2009; 113:1201-1203). Briefly, mMSCs or hMSCs were plated at a density of 1×10⁴ cells/cm² in 6-well plates or T75 flasks (Falcon, Fisher Scientific) and incubated overnight. The cells were counted and then exposed to fresh culture medium containing Ad5CMVntlacZ at 0, 300, or 2,000 MOI for 48 hours. The cells were then X-gal stained for β-galactosidase activity and the blue nuclear-targeted β-galactosidase positive Ad5CMVntlacZ-transduced mMSCs or hMSCs (ntlacZ-mMSCs or ntlacZ-hMSCs) were identified. To conduct X-gal cytochemistry staining for β-galactosidase activity, ntlacZ-mMSCs or ntlacZ-hMSCs were washed with PBS, fixed for 5 min in fixing solution (2% formaldehyde, 0.2% glutaraldehyde, prepared in PBS, Sigma, St. Louis, Mo.), washed twice with PBS, and incubated in staining solution (1 mg/ml X-gal, 5 mM K ferricyanide, 5 mM K ferrocyanide, and 2 mM MgCl2, prepared in PBS, Sigma) at 37° C. in the dark overnight. Cells were washed with PBS and the expression of transgene ntlacZ in ntlacZ-mMSCs or ntlacZ-hMSCs was evaluated by light microscopy (Nikon Eclipse TS 100 Microscope, Nikon Inc., Melville, N.Y.) scoring of blue cells expressing the nuclear-targeted β-galactosidase activity (Deng et al., Stem Cells. 2004; 22:1279-1291; Abdel-Mageed et al., Blood. 2009; 113:1201-1203).

Persistence of Adenoviral-Mediated Transgene Expression in mMSCs in Culture

Persistence of adenoviral-mediated transgene expression in mMSCs was determined in vitro using our previously described method (Deng et al., Stem Cells. 2004; 22:1279-1291). To study the persistence of adenoviral-mediated secretion of biologically active ECSOD in vitro, mMSCs were transduced with Ad5CMVECSOD at MOI 2,000 for 48 hours. The virus-containing culture medium was removed, and the cells were washed 3 times with PBS. The cells were then counted, cultured in fresh culture medium for 48 hours, and culture supernatant was collected. The cells were further incubated in fresh culture medium containing 2% FBS, and the medium was changed every 2-3 days until day 35. The cells were then cultured in fresh culture medium for 48 hours, and culture supernatant was collected. The 48-hour culture supernatant at day 0 and 35 after transduction were then analyzed for ECSOD secretion using a SOD activity assay kit (Cayman Chemical Company). Each value represents mean±SEM (n=3). To study the persistence of ntlacZ transgene expression in vitro, mMSCs were transduced with Ad5CMVntlacZ at MOI 2,000 for 48 hours. The virus-containing culture medium was removed, and the cells were washed 3 times with PBS. Some cells were X-gal stained for β-galactosidase activity and the blue nuclear-targeted β-galactosidase positive ntlacZ-mMSCs were identified. Other cells were further incubated in fresh culture medium containing 2% FBS, and the medium was changed every 2-3 days until day 35. The cells were then X-gal stained for β-galactosidase activity and the blue nuclear-targeted β-galactosidase positive ntlacZ-mMSCs were identified.

Intravenous Administration of ECSOD-mMSCs into Irradiated Mice Through Tail Vein Injection and Animal Health Observations

Five-week-old female BALB/c mice (The Jackson Laboratory) were given 9 Gy total body γ irradiation from a ¹³⁷Cs source (Gammacell 1000, Model B; MDS Nordion, Ottawa, ON, Canada) at a dose rate of 1.23 Gy/min. Twenty four hours later, these animals received a tail vein injection of 200 μl PBS, 0.5×10⁶ ntlacZ-mMSCs in 200 μl PBS, or 0.5×10⁶ ECSOD-mMSCs in 200 μl PBS. All in vivo experiments were performed on mice in accordance with institutional and NIH guidelines for the care and use of laboratory animals. To prepare ntlacZ-mMSCs or ECSOD-mMSCs, mMSCs were transduced with Ad5CMVntlacZ or Ad5CMVECSOD at MOI 2,000 for 48 hours. The virus-containing culture medium was removed and the cells were washed 3 times with PBS. The cells were then harvested with 0.25% Trypsin/1 mM EDTA for 2 minutes at 37° C., washed twice with PBS, and a cell suspension at a concentration of 2.5×10⁶ cells/ml was prepared in PBS. For intravenous administration of PBS, ntlacZ-mMSCs, or ECSOD-mMSCs into the irradiated mice, 200 μl of PBS or 200 μl of cell suspension were injected into the tail vein using a 27-gauge needle (Abdel-Mageed et al., Blood. 2009; 113:1201-1203). A total of 0.5×10⁶ cells or 200 μl PBS were injected into each mouse. The mice were then monitored for survival, cataract formation, and carcinogenesis over the whole lifespan. Kaplan-Meier survival curve was used for data analysis and statistical significance was determined using logrank test. P<0.05 was considered statistically significant.

The results of the experimental examples are now discussed.

### Improvement of Survival in Irradiated Mice by Intravenous Administration of ECSOD-MSC

Mesenchymal stem cells (MSCs) are multipotent adult stem cells from bone marrow (Prockop, Science. 1997; 276:71-74. Pittenger et al., Science. 1999; 284:143-147. Deng et al., Biochem Biophys Res Commun. 2001; 282:148-152; Sun et al, Stem Cells 2003; 21:527-535. Peister et al, Blood 2004; 103:1662-1668). These cells have advantages over other stem cells in that they can be easily isolated from patients or donors, readily expanded ex vivo, and efficiently gene engineered. Therefore, MSCs hold promise as vehicles for adult stem cell-based gene therapy (Bivalacqua et al., Am J Physiol Heart Circ Physiol 2007; 292:H1278-1290).

Mice given 9 to 10 Gy total body irradiation die a hematologic death 10 to 14 days after exposure (Millar et al., Int J Radiat Oncol Biol Phys 1982; 8:581-583). It has been found that MSCs migrate to radiation-injured tissues such as bone marrow, gastrointestinal tract, skin, and muscle after intravenous administration (Chapel et al., J Gene Med 2003; 5:1028-1038). Therefore, MSCs hold promise as vehicles for adult stem cell-based gene therapy to improve survival after radiation exposure. To develop an improved therapy using adult stem cells, we hypothesize that MSCs can be used for ex vivo ECSOD gene therapy to improve survival after radiation exposure.

To test our hypothesis that intravenous administration of MSC that secretes biologically active ECSOD enzyme (ECSOD-MSC) can improve survival after radiation exposure, mouse MSCs (mMSCs) were isolated by their adherence to tissue-culture plastic from 6-week-old female BALB/c mice and ex vivo expanded (Abdel-Mageed et al., Blood. 2009; 113:1201-1203). The cells were differentiated into osteoblasts and adipocytes in vitro, and cell phenotype was analyzed by flow cytometry. FIG. 1A shows that the cells express CD105, CD44, CD29, stem cell antigen-1 (Sca-1), and CD13. The cells do not express CD11b, CD34, CD45, CD19, CD31, CD117 (c-Kit), CD135, CD90 (Thy-1.2), or CD73. Therefore, these cells are typical MSCs (Sun et al, Stem Cells 2003; 21:527-535. Peister et al, Blood 2004; 103:1662-1668; Abdel-Mageed et al., Blood. 2009; 113:1201-1203).

mMSCs were then transduced with Ad5CMVECSOD, an adenovirus carrying human ECSOD gene under the control of cytomegalovirus (CMV) promoter (Chu et al., Circ Res 2003; 92:461-468), and culture supernatant was analyzed for superoxide dismutase (SOD) activity. FIG. 1B demonstrates a dose-dependent secretion of high level biologically active ECSOD by Ad5CMVECSOD-transduced mMSCs. Therefore, high level biologically active ECSOD enzyme secretion by mMSCs can be achieved after adenoviral-mediated gene transfer. mMSCs were further transduced with Ad5CMVntlacZ, an adenovirus carrying nuclear-targeted 3-galactosidase gene ntlacZ under the control of CMV promoter (Chu et al., Circ Res 2003; 92:461-468), and analyzed by X-gal staining. As shown in FIG. 1C, transduction efficiency is dose-dependent.

To determine whether intravenous administration of mouse MSC that secretes biologically active ECSOD enzyme (ECSOD-mMSC) can improve survival after radiation exposure, a mouse model of lethal total body γ radiation was used. Briefly, 5-week-old female BALB/c mice were given 9 Gy total body γ irradiation from a ¹³⁷Cs source. Twenty-four hours later, the animals were given a tail vein injection of phosphate-buffered saline (PBS), Ad5CMVntlacZ-transduced mMSCs (ntlacZ-mMSCs), or Ad5CMVECSOD-transduced mMSCs (ECSOD-mMSCs). As shown in FIG. 1D, 52% of animals in the ECSOD-mMSC treatment group survived for 35 days, whereas only 9% of animals in the ntlacZ-mMSC treatment group and 10% of animals in the PBS treatment group survived for 35 days. Furthermore, all mice that survived for 35 days also survived for 5 months. These findings demonstrate for the first time that intravenous administration of ECSOD-MSC improves survival in irradiated mice from 10% to 52% (Abdel-Mageed et al., Blood. 2009; 113:1201-1203), highlighting its clinical potential to improve survival of patients with radiation exposure resulting from nuclear war, nuclear accident, nuclear terrorism, and other radiologic emergencies.

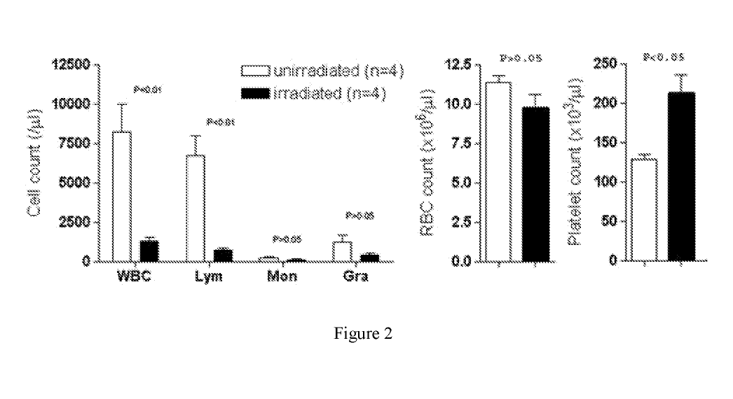

Mice given 9 to 10 Gy total body irradiation die a hematologic death 10 to 14 days after exposure (Millar et al., Int J Radiat Oncol Biol Phys 1982; 8:581-583). To investigate whether radiation induces bone marrow failure in mice in our study, 5-week-old female BALB/c mice were given 9 Gy total body γ irradiation from a ¹³⁷Cs source. Seven days later, the mice were euthanized and peripheral blood and bone marrow were analyzed. Complete blood count (CBC) showed that white blood cell (WBC) and lymphocyte counts in peripheral blood of the irradiated mice decreased (FIG. 2). The number of nucleated bone marrow cells of the femur of irradiated mice also decreased. Therefore, γ radiation causes bone marrow failure in mice in our study.

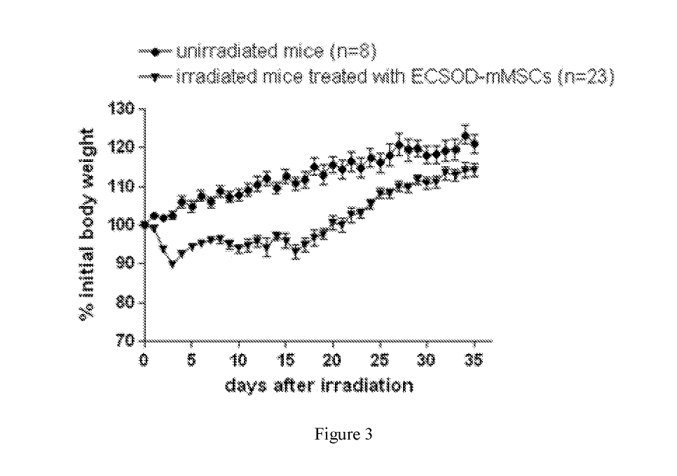

To investigate the effect of intravenous administration of ECSOD-mMSCs on mouse body weight loss after radiation exposure, all of the mice are monitored daily and their body weights are recorded daily for 35 days. In our study, the most common symptom is loss of body weight, followed by slow, shallow, labored breathing, hunched posture, ruffled fur, failure to groom (goes along with hunched posture), lethargy, self-segregation (goes along with lethargy), and inability to eat or drink. But mice in ECSOD-mMSCs treatment group started to gain weight around 16 days after irradiation (FIG. 3). Therefore, γ radiation might have caused gastrointestinal tract injury in our study.

It has been found that the cells of the body that are most vulnerable to damage from radiation are the rapidly dividing cells of the bone marrow and the intestinal lining (Mettler and Voelz, N Engl J Med. 2002; 346:1554-61; Zenk J L. Expert Opin Investig Drugs. 2007; 16:767-70). It has also been found that MSCs migrate to radiation-injured tissues such as bone marrow and gastrointestinal tract after intravenous administration (Chapel et al., J Gene Med 2003; 5:1028-1038). Therefore, the improvement in survival of irradiated mice might result from the scavenger of O₂⁻in the irradiated tissues such as bone marrow and gastrointestinal tract by ECSOD secreted from Ad5CMVECSOD-transduced MSCs.

Therefore, our novel method harnesses the power of migration of MSCs to radiation-injured tissues such as bone marrow and gastrointestinal tract after intravenous administration and adenovirus-mediated ECSOD gene therapy to improve survival after radiation exposure (Millar et al., Int J Radiat Oncol Biol Phys 1982; 8:581-583. Chapel et al., J Gene Med 2003; 5:1028-1038). These findings suggest that this novel adult stem cell-based ex vivo gene therapy, i.e. intravenous administration of ECSOD-MSCs to a patient with radiation exposure, may represent a new form of therapy to improve survival after radiation exposure.

### Mitigation of Delayed Effects of Acute Radiation Exposure in Irradiated Mice by Intravenous Administration of ECSOD-MSC

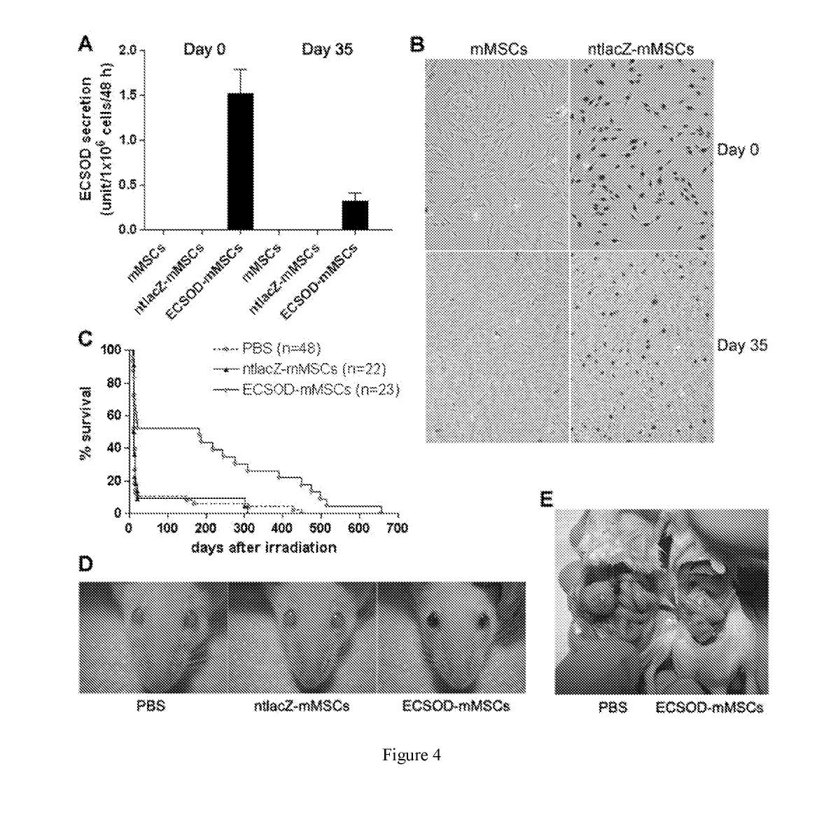

Exposure to high doses of ionizing radiation can lead to death, lifespan shortening, cataract formation, or carcinogenesis (Millar et al., Int J Radiat Oncol Biol Phys 1982; 8:581-583; Epperly et al., Radiat Res. 2008; 170:437-443; Orr and Sohal. Science. 1994; 263:1128-1130; Lin et al., Mol Vis. 2005; 11:853-858; Hall and Henry. Int J Radiat Biol. 2004; 80:327-337). To study the persistence of ECSOD transgene expression in vitro, mMSCs were transduced with Ad5CMVECSOD and the cells were then cultured for 35 days. As shown in FIG. 4A, ECSOD-mMSCs secreted 1.52±0.27 unit/1×10⁶ cells/48 hour (mean±SEM, n=3) ECSOD at day 0 and 0.32±0.09 unit/1×10⁶ cells/48 hour (mean±SEM, n=3) ECSOD at day 35. To study the persistence of ntlacZ transgene expression in vitro, mMSCs were transduced with Ad5CMVntlacZ and the cells were then cultured for 35 days. As shown in FIG. 4B, the percentage of cells expressing β-galactosidase was 99%±1 (mean±SEM, n=3) at day 0 and 24%±5 (mean±SEM, n=3) at day 35. Therefore, adenoviral-mediated transgene expression in mMSCs can persist for more than 35 days in culture.

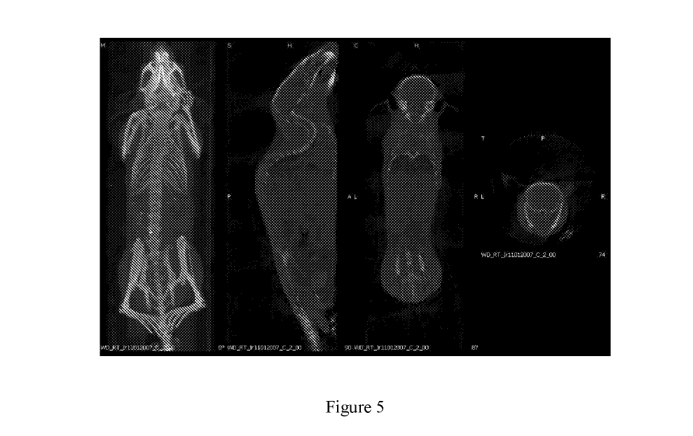

To determine whether intravenous administration of ECSOD-MSCs can mitigate delayed effects of acute radiation exposure such as lifespan shortening, cataractogenesis, and carcinogenesis (Epperly et al., Radiat Res. 2008; 170:437-443; On and Sohal. Science. 1994; 263:1128-1130; Lin et al., Mol Vis. 2005; 11:853-858; Hall and Henry. Int J Radiat Biol. 2004; 80:327-337), mice that had survived for 35 days were then monitored for survival, cataract formation, and carcinogenesis over their remaining lifespan. Previous studies have demonstrated that overexpression of superoxide dismutase extends lifespan in Drosophila (Orr and Sohal. Science. 1994; 263:1128-1130) and prevents cataract formation in rats (Lin et al., Mol Vis. 2005; 11:853-858). In our study, irradiated mice had a shortened lifespan. However, mice in the ECSOD-mMSCs treatment group survived 207 days longer than mice in the PBS or ntlacZ-mMSCs treatment group (FIG. 4C). Mice in the ECSOD-mMSCs treatment group developed cataracts 39 days later than mice in the PBS or ntlacZ-mMSCs treatment group (FIG. 4D). No tumor development was observed in mice in the ECSOD-mMSCs treatment group, whereas large abdominal tumor was found in mice in the PBS treatment group (FIG. 4E). Further, no new bone formation was observed in irradiated mice treated with ECSOD-mMSCs (FIG. 5), suggesting the safety of ECSOD-MSCs treatment. Therefore, mitigation of both acute radiation syndrome and delayed effects of acute radiation exposure has been successfully achieved by intravenous administration of ECSOD-MSCs at 24 hours after radiation exposure in mice. The mechanism might be that scavenging of O₂⁻in the extracellular space of irradiated tissues by ECSOD secreted from ECSOD-MSCs that have migrated to radiation-injured tissues after intravenous administration can prevent further tissue injuries.

Adenoviral-Mediated ECSOD Gene Transfer into Human MSC

For the first stage in clinical proof-of-concept, human MSCs (hMSCs) were isolated by their adherence to tissue culture plastic from 42 healthy bone marrow donors and ex vivo expanded as previously described (Prockop, Science. 1997; 276:71-74. Pittenger et al., Science. 1999; 284:143-147; Deng et al., Biochem Biophys Res Commun. 2001; 282:148-152). FIG. 6A shows colony-forming unit (CFU) of hMSCs in 14-day cultures of human bone marrow mononuclear cells (BM-MNCs). No CFU of hMSCs was identified in 14-day cultures of human peripheral blood mononuclear cells (PB-MNCs). Quantification of CFU assay demonstrated that 8±1 (mean±SEM, n=42) colonies of hMSCs were derived from 1×10⁵ human BM-MNCs (FIG. 6B), similar to a previous study by other investigators (Pittenger et al., Science. 1999; 284:143-147). The cells were then differentiated into osteoblasts and adipocytes in vitro and cell phenotype was analyzed by flow cytometry. FIG. 6C shows that the cells express CD105, CD73, CD90, CD29, and CD44. The cells do not express CD45, CD34, CD14, Lin1, and HLA-DR. Therefore, these cells are typical MSCs (Dominici et al., Cytotherapy. 2006; 8:315-317). Cytogenetic analysis of the cells was conducted using a spectral karyotyping (SKY) assay. FIG. 6D shows cells with a normal human karyotype. Therefore, there is no acquisition of cytogenetic abnormalities after ex vivo expansion of hMSCs.

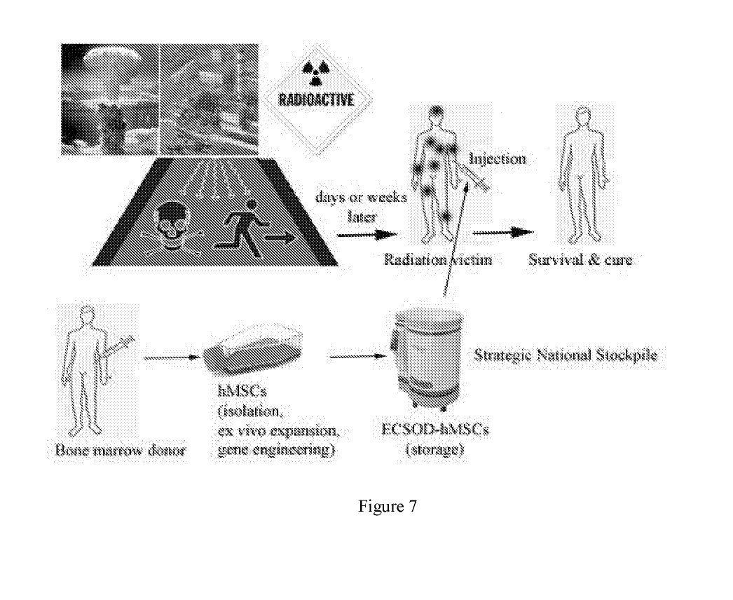

To study the efficacy of adenoviral-mediated transgene expression in hMSCs, hMSCs were transduced with Ad5CMVntlacZ and then analyzed by X-gal staining for nuclear-targeted β-galactosidase activity. As shown in FIG. 6E, adenoviral-mediated ntlacZ transgene expression in Ad5CMVntlacZ-transduced hMSCs (ntlacZ-hMSCs) is dose-dependent. hMSCs were further transduced with Ad5CMVECSOD and the culture supernatant was analyzed for superoxide dismutase activity. FIG. 6F demonstrates a dose-dependent, high-level secretion of biologically active ECSOD by Ad5CMVECSOD-transduced hMSCs (ECSOD-hMSCs). Therefore, hMSCs can be genetically modified with adenoviral vector to secrete high levels of biologically active ECSOD enzyme. Our findings suggest that mesenchymal stem cell-based antioxidant gene therapy has the potential for treatment of radiation exposure in humans as a consequence of radiological and nuclear emergencies, space radiation exposure, and cancer radiation diagnosis and radiotherapy toxicity (FIG. 7).

### REFERENCES

1. Abdel-Mageed A S, Senagore A J, Pietryga D W, et al. Intravenous administration of mesenchymal stem cells genetically modified with extracellular superoxide dismutase improves survival in irradiated mice. Blood. 2009; 113(5):1201-1203.
2. Baber S R, Deng W, Master R G, et al. Intratracheal mesenchymal stem cell administration attenuates monocrotaline-induced pulmonary hypertension and endothelial dysfunction. Am J Physiol Heart Circ Physiol. 2007; 292(2): H1120-1128.
3. Bivalacqua T J, Deng W, Kendirci M, et al. Mesenchymal stem cells alone or ex vivo gene modified with endothelial nitric oxide synthase reverse age-associated erectile dysfunction. Am J Physiol Heart Circ Physiol. 2007; 292(3):H1278-1290.
4. Bivalacqua T J, Usta M F, Kendirci M, et al. Superoxide anion production in the rat penis impairs erectile function in diabetes: influence of in vivo extracellular superoxide dismutase gene therapy. J Sex Med. 2005; 2(2):187-197.
5. Chapel A, Bertho J M, Bensidhoum M, et al. Mesenchymal stem cells home to injured tissues when co-infused with hematopoietic cells to treat a radiation-induced multi-organ failure syndrome. J Gene Med. 2003; 5(12):1028-1038.
6. Chu Y, Iida S, Lund D D, et al. Gene transfer of extracellular superoxide dismutase reduces arterial pressure in spontaneously hypertensive rats: role of heparin-binding domain. Circ Res. 2003; 92(4):461-468.
7. Deng W, Bivalacqua T J, Champion H C, et al. Superoxide dismutase—a target for gene therapeutic approach to reduce oxidative stress in erectile dysfunction. Methods Mol Biol. 2010; 610:213-227.
8. Deng W, Bivalacqua T J, Chattergoon N N, et al. Adenoviral gene transfer of endothelial nitric oxide synthase: High level expression in ex vivo expanded marrow stromal cells. Am J Physiol Cell Physiol. 2003; 285(5): C1322-1329.
9. Deng W, Bivalacqua T J, Chattergoon N N, et al. Engineering ex vivo-expanded marrow stromal cells to secrete calcitonin gene-related peptide using adenoviral vector. Stem Cells. 2004; 22(7): 1279-1291.
10. Deng W, St Hilaire R C, Chattergoon N N, et al. Inhibition of vascular smooth muscle cell proliferation in vitro by genetically engineered marrow stromal cells secreting calcitonin gene-related peptide. Life Sci 2006; 78(16): 1830-8.
11. Deng W, Obrocka M, Fischer I, et al. In vitro differentiation of human marrow stromal cells into early progenitors of neural cells by conditions that increase intracellular cyclic AMP. Biochem Biophys Res Commun. 2001; 282(1):148-152.
12. Dominici M, Le Blanc K, Mueller I, et al. Minimal criteria for defining multipotent mesenchymal stromal cells. The International Society for Cellular Therapy position statement. Cytotherapy. 2006; 8(4):315-317.
13. Epperly M W, Dixon T, Wang H, et al. Modulation of radiation-induced life shortening by systemic intravenous MnSOD-plasmid liposome gene therapy. Radiat Res. 2008; 170(4):437-443.
14. Fliedner T M. Nuclear terrorism: the role of hematology in coping with its health consequences. Curr Opin Hematol. 2006; 13(6):436-444.
15. Greenberger J S, Epperly M W. Pleiotrophic stem cell and tissue effects of ionizing irradiation protection by MnSOD-plasmid liposome gene therapy. In: Columbus Frank, Ed. Progress in Gene Therapy. New York City, N.Y.: Nova Science Publications; 2005:110-118.
16. Greenberger J S. Could gene therapy prevent the effects of irradiation exposure? Pharmacogenomics. 2006; 7(8):1141-1145.
17. Hall E J. Henry S. Kaplan Distinguished Scientist Award 2003. The crooked shall be made straight; dose-response relationships for carcinogenesis. Int J Radiat Biol. 2004; 80(5):327-337.
18. Lin D, Barnett M, Grauer L, et al. Expression of superoxide dismutase in whole lens prevents cataract formation. Mol Vis. 2005; 11:853-858.
19. Mettler F A Jr, Voelz G L. Major radiation exposure—what to expect and how to respond. N Engl J Med. 2002; 346(20):1554-61.
20. Millar J L, Stephens T C, Wist E A. An explanation for the ability of cytotoxic drug pretreatment to reduce bone marrow related lethality of total body irradiation (TBI). Int J Radiat Oncol Biol Phys. 1982; 8(3-4):581-583.
21. Orr W C, Sohal R S. Extension of life-span by overexpression of superoxide dismutase and catalase in Drosophila melanogaster. Science. 1994; 263(5150):1128-1130.
22. Peister A, Mellad J A, Larson B L, et al. Adult stem cells from bone marrow (MSCs) isolated from different strains of inbred mice vary in surface epitopes, rates of proliferation, and differentiation potential. Blood. 2004; 103(5):1662-1668.
23. Peterson S E, Westra J W, Paczkowski C M, et al. Chromosomal mosaicism in neural stem cells. Methods Mol Biol. 2008; 438:197-204.
24. Pittenger M F, Mackay A M, Beck S C, et al. Multilineage potential of adult human mesenchymal stem cells. Science. 1999; 284(5411):143-147.
25. Prockop D J. Marrow stromal cells as stem cells for nonhematopoietic tissues. Science. 1997; 276(5309):71-4.
26. Sun S, Guo Z, Xiao X, et al. Isolation of mouse marrow mesenchymal progenitors by a novel and reliable method. Stem Cells. 2003; 21(5):527-535.
27. Zenk J L. New therapy for the prevention and prophylactic treatment of acute radiation syndrome. Expert Opin Investig Drugs. 2007; 16(6):767-70.

Whereas this invention has been disclosed with reference to specific embodiments, it is apparent that other embodiments and variations of this invention can be devised by other people skilled in the art without departing from the true spirit and scope of this invention. Therefore, the appended claims include all such embodiments and equivalent variations in accordance with the principals of patent law.

We claim:

1. A method of improving survival after radiation exposure, said method comprising an adult stem cell-based gene delivery system, wherein the adult stem cell is bone marrow mesenchymal stem cell (MSC) and the gene is extracellular superoxide dismutase (ECSOD) gene.

2. The method of claim 1, wherein said MSC is human.

3. The method of claim 1, wherein said ECSOD is human.

4. (canceled)

5. (canceled)

6. (canceled)

7. A method of intravenous administration of ECSOD-MSCs for improving survival after radiation exposure, said method comprising intravenous administration of ECSOD-MSCs to a patient with radiation exposure, wherein ECSOD-MSCs migrate to radiation-injured tissues such as bone marrow and gastrointestinal tract after intravenous administration, locally secrete biologically active ECSOD enzyme, and the secreted ECSOD enzyme scavenges superoxide anion (O2−) in radiation-injured tissues, thereby improving survival of patients with radiation exposure.

8. The method of claim 7, wherein said patient is human.

9. The method of claim 7, wherein said radiation is γ radiation from nuclear war, nuclear accident, nuclear terrorism, radiation diagnosis, radiation therapy, radiological emergencies, and space travel.
